(19) United States
(12) Patent Application Publication
(10) Pub. No.: US 2024/0298764 A1
(43) Pub. Date: Sep. 12, 2024

(54) PORTABLE HOME CARE ORGANIZATION SYSTEM AND METHOD

(71) Applicant: Lynn-Marie Sharp Rivera, Rancho Mission Viejo, CA (US)

(72) Inventor: Lynn-Marie Sharp Rivera, Rancho Mission Viejo, CA (US)

(21) Appl. No.: 18/598,534

(22) Filed: Mar. 7, 2024

Related U.S. Application Data
(60) Provisional application No. 63/450,880, filed on Mar. 8, 2023.

(51) Int. Cl. A45C 9/00; A45C 5/03; A45C 5/06; A45C 5/14; A45C 13/02; A45C 13/26; A61B 50/30

(52) U.S. Cl. CPC A45C 9/00; A45C 5/03; A45C 5/065; A45C 5/145; A45C 13/02; A45C 13/262; A61B 50/30; A45C 2013/025; A45C 2013/267; A61B 2050/3008

(57) ABSTRACT

A home care organization system includes a housing, an upper assembly comprising a first compartment, wherein the first compartment is configured to store clean equipment and material, a platform configured to be movable from a vertical to a horizontal orientation relative to the upper assembly, wherein the platform is laterally extendable to provide a first surface configured to receive clean equipment and material and a second surface configured to receive dirty equipment and material, and a lower assembly.

### FIELD OF THE INVENTION

This application claims the benefit of U.S. Provisional Patent Application No. 63/450,880 filed on Mar. 8, 2023, the entirety of which is incorporated by reference herein.

### FIELD OF THE INVENTION

The present invention relates generally to organization systems, and more particularly, to portable home care organization systems and methods.

### BACKGROUND OF THE INVENTION

Organization systems may include a series of compartments configured to store different types of materials or objects. While these systems may provide the ability to segregate materials or objects from each other, they are not optimal for use in the home care medical field where strict procedures and processes must be followed to protect the safety and health of a patient. Current systems utilized in a patient's home are insufficient, including because they rely on a patient's home surfaces (e.g., tables, chairs, countertops, furniture, personal belongings, etc.) to assist the practitioner in administering the home care. One major concern in using patient's home surfaces is cleanliness, sterility, and lack of contaminants, particularly when many home care patients are very limited in their function, having multiple comorbidities, infections, or await pending tests due to suspected infection and are most likely unable to tolerate or have the energy or prioritize cleaning the home sufficiently for use by a medical practitioner in the home.

A bag technique using a home healthcare bag has been developed to establish a clean work area by finding a surface in a home to prevent contamination of bag and equipment, avoid cross infection to protect patients, their families, and healthcare providers while care is provided for patients in the home. The home healthcare bag is brought into the home by the caregiver. The bag includes compartments made of hard, non-porous surfaces that can be disinfected by wiping the surface to clean and disinfect the exterior. The bag technique requires the home care provider to select a clean, flat, hard surface to place the home healthcare bag and set up the work area using discretion and consideration when placing a bag on a patient's furniture or other surface. It is never recommended to place the bag on the floor. When not in use, the home healthcare bag must be closed and fastened during a visit. A barrier must be placed under the bag and the patient's furniture or surface to help prevent transmission of infection. In practice, items such as blood pressure cuffs, stethoscopes, documents for patient are placed at one corner of the work area on a barrier. Another barrier is needed underneath a receptacle plastic trash bag for disposable items. Once care is administered, reusable items such as the blood pressure cuffs, stethoscope, etc., are placed on the receptacle trash bag. Disposable sharp items are separately kept in a disposable sharps container and, after use, kept in a separate “dirty” compartment.

Reusable items must be cleaned and disinfected prior to returning to the bag. Multiple disinfecting wipes may take 2-5 minutes to let stand and dry completely prior to placing in the bag. Once the items are clean, they are placed back onto the clean barrier again to dry prior to putting in the bag.

The bag technique typically requires three surfaces with barriers; large surfaces are needed if a computer/tablet is also used with a barrier. A first surface is required for the home healthcare bag. A second surface is required for clean, disposable, and reusable equipment prior to touching the patient. A third surface is required for used and discarded supplies/equipment after they touch the patient; after cleaning, they are then placed on the clean surface for 2-5 minutes to dry before placing the items back in the home healthcare bag. If there is no visibly clean, dry surface available in the home, it is expected to place a barrier under the bag or hang it on a door knob or back of chair, and makes transferring of equipment on an unknown surface difficult. Use of a heavy chair, door handle, or other like surfaces in the home are not preferred and present challenges to administering care to a patient in their home.

Further, requiring the use of patients' home surfaces may have a significant impact psychologically. During the COVID-19 pandemic, high stress and increased psychological disabilities such as OCD and anxiety exacerbated the patients' concern of contact between their personal belongings and the clinicians personal belongings. Additional known or unknown risks include infestation of pests, roaches, fleas, feces, or urine from animals, and/or humans with incontinence issues, bedbugs, etc. Many of these systems are not portable or not configured to be brought into a patient's home. Therefore, there exists a need for a portable system of compartments and features configured to meet the strict procedures and processes in the home care medical field to protect the safety and health of patients in their home.

The background description disclosed anywhere in this patent application includes information that may be useful in understanding the present invention. It is not an admission that any of the information provided herein is prior art or relevant to the presently claimed invention, or that any publication specifically or implicitly referenced is prior art.

### SUMMARY OF THE PREFERRED EMBODIMENTS

In accordance with an aspect of the present invention there is provided a home care organization system that includes a housing, an upper assembly comprising a first compartment, wherein the first compartment is configured to store clean equipment and material, a platform configured to be movable from a vertical to a horizontal orientation relative to the upper assembly, wherein the platform is laterally extendable to provide a first surface configured to receive clean equipment and material and a second surface configured to receive dirty equipment and material, and a lower assembly.

The first compartment may be sealable. The first compartment may be segregated into a first partition and a second partition, the first partition configured to store the clean material and the second partition configured to store the clean equipment. The upper assembly may include a second compartment configured to store the clean equipment, and wherein the first compartment is configured to store the clean material. The upper assembly may also include a locking mechanism such that only one of the first compartment or the second compartment is able to be opened at one time. The housing may include a handle configured to expand the upper assembly vertically to position the platform at a specified height from the bottom of the housing.

The clean equipment may include one or more of a blood pressure cuff, a stethoscope, a bandage scissors, a reusable tourniquet, a thermometer, a pulse oximeter, and a writing instrument. The clean material may include one or more of wipes, gloves, barriers, single-use disposable items, syringes, needles, masks, and personal protective equipment.

The upper assembly may further include a hinge configured to move the platform from the vertical to the horizontal position and a locking mechanism configured to secure the platform in the horizontal position. The lower assembly may include a third compartment configured to store one or more of patient files, a personal computing device, and a printer. The system may further include a lock configured to secure the third compartment. The housing may include a front portion and a rear portion, wherein the rear portion comprises a third compartment configured to store dirty materials. The upper assembly may include a fourth compartment configured to swivel out laterally from the upper assembly and to receive one or more of hand sanitizer and soap. The upper assembly may include a fifth compartment configured to store dirty material. The upper assembly may include a sixth compartment configured to store a sharps container. The system may further include wheels affixed to the lower assembly. The wheels may be lockable. The platform may be configured to receive a personal computing device.

In accordance with an aspect of the present invention there is provided a home care organization method. A home care organization system is provided including a housing, an upper assembly comprising first and second compartments, wherein the first compartment is configured to store clean material and the second compartment is configured to store clean equipment, a platform configured to be movable from a vertical to a horizontal orientation relative to the upper assembly, wherein the platform is laterally extendable to provide a first surface configured to receive clean equipment and material and a second surface configured to receive dirty equipment and material, and a lower assembly, wherein the lower assembly comprises a third compartment configured to store patient files and a personal computing device. The system is expanded vertically to expose the platform. The platform is moved from a vertical to a horizontal orientation relative to the upper assembly. The platform is expanded laterally to provide the first surface and the second surface. The personal computing device is removed and placed on the platform proximate to the first and second surfaces. Home care services are provided to a patient using the clean material and equipment, the platform, including the first and second surfaces, and the personal computing device.

The first compartment may be opened to access the clean materials, the clean material placed on the first surface in accordance with the home care services to be administered to the patient, the first compartment is closed, and the hands used to provide the home care services are cleaned and sanitized.

The second compartment may be opened to access the clean equipment, the clean equipment placed on the first surface in accordance with the home care services to be administered to the patient, the second compartment is closed, and the hands used to provide the home care services are cleaned and sanitized.

When the home care services are provided to the patient, used material and equipment may be placed on the second surface to isolate the used material and equipment from the clean material and equipment.

Further, the used equipment may be cleaned and sanitized, the cleaned and sanitized equipment may be placed on first surface, the cleaned and sanitized equipment may be replaced in the second compartment, and the used material may be discarded in a fourth compartment configured to handle waste,

Information documenting the home care services may be imputed using the personal computing device.

A signature of the patient consenting to the home care services may be obtained using a personal computing device, the personal computing device may be cleaned and sanitized, and the personal computing device may be replaced in the third container.

The clean material may include at least one of a syringe and needle, and wherein when the at least one of the syringe and needle are used, placing the used syringe and needle into a sharps container.

Hand sanitizer and soap may be placed in a holder rotatably connected to the upper assembly.

### BRIEF DESCRIPTION OF THE DRAWINGS

The invention may be more readily understood by referring to the accompanying drawings in which:

FIG. 1 is a front view diagram of a portable home care organization system in accordance with a preferred embodiment of the present invention;

FIG. 2 is a side view diagram of a portable home care organization system in accordance with a preferred embodiment of the present invention;

FIG. 3 is a rear view diagram of a portable home care organization system in accordance with a preferred embodiment of the present invention;

FIG. 4 is a perspective view of a partially opened home care organization system in accordance with a preferred embodiment of the present invention;

FIG. 5 is a perspective view of a partially opened home care organization system in accordance with a preferred embodiment of the present invention;

FIG. 6 is a flowchart of a home care organization method in accordance with a preferred embodiment of the present invention;

FIG. 7 is a flowchart of a home care organization method in accordance with a preferred embodiment of the present invention; and

FIG. 8 is a flowchart of a home care organization method in accordance with a preferred embodiment of the present invention.

Like numerals refer to like parts throughout the several views of the drawings.

### DETAILED DESCRIPTION OF THE PREFERRED EMBODIMENTS

The following description and drawings are illustrative and are not to be construed as limiting. Numerous specific details are described to provide a thorough understanding of the disclosure. However, in certain instances, well-known or conventional details are not described in order to avoid obscuring the description. References to one or another embodiment in the present disclosure can be, but not necessarily are, references to the same embodiment; and, such references mean at least one of the embodiments.

Reference in this specification to “one embodiment” or “an embodiment” means that a particular feature, structure, or characteristic described in connection with the embodiment is included in at least one embodiment of the disclosure. Appearances of the phrase “in one embodiment” in various places in the specification do not necessarily refer to the same embodiment, nor are separate or alternative embodiments mutually exclusive of other embodiments. Moreover, various features are described which may be exhibited by some embodiments and not by others. Similarly, various requirements are described which may be requirements for some embodiments but not for other embodiments.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks: The use of highlighting has no influence on the scope and meaning of a term; the scope and meaning of a term is the same, in the same context, whether or not it is highlighted. It will be appreciated that the same thing can be said in more than one way.

Consequently, alternative language and synonyms may be used for any one or more of the terms discussed herein. Nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

It will be appreciated that terms such as “front,” “back,” “top,” “bottom,” “side,” “short,” “long,” “up,” “down,” and “below” used herein are merely for ease of description and refer to the orientation of the components as shown in the figures. It should be understood that any orientation of the components described herein is within the scope of the present invention.

The home care organization system and method disclosed herein include surfaces attached to the system so that the practitioner or clinician does not need to use patient surfaces to administer home care services. In an embodiment, the system includes three adjustable surfaces to place clean barriers for placement of a laptop on a surface, a separate surface for clean equipment, material, and supplies, and yet another separate surface for used “dirty” equipment, material and supplies used on the patient in administering the home care services. The surfaces of the system are adjustable to allow height variations that may depend on the clinician's height and allow for safe and proper body mechanics and posture with decreased risk of workplace injury. The adjustable surfaces allow for all equipment, material, and supplies to be easily, quickly, and efficiently placed on surfaces without requiring extra time administering the home care services or impede on a patient's personal belongings or surfaces. The system preferably includes wheels to enable the clinician to wheel the system into the home, and a strap or locking device to prevent the system from moving or rolling during administration of the home care services.

The system is designed to include everything that is needed in the home to provide total care, greater independence, efficiency, and professionalism and overall increased time to focus on assessment and treatment without unnecessary time to find adequate workspace that may not be an acceptable environment.

In preferred embodiments, the system and method allows traveling between multiple patients without ever touching patient's furniture. This helps provide increased control of transmission of pathogens in contact between the clinician's system, including the equipment, material, and supplies, and the home or patient and reduces the need for barriers to protect the system from pathogens present in the home. The system with adjustable surfaces and multiple compartments in the same system decreases risk to exposure to pathogens and reduces risk of transmission from home to system and patient to patient.

Thus, the system and method provides more efficient home care services by not having to find a surface in the home, decreases risk and prevention of cross-contamination and transmission of pathogens, and provides greater ease and comfort to the patient and caregiver who enters the home.

Lipincott Procedures (e.g., Lipincott's Nursing Procedures) set forth best practices and requirements for in-home care nursing and medical practitioners to ensure safety, sterility, and appropriate treatment of sterile and non-sterile equipment and materials through segregation into various compartments (e.g., bag compartments) and disinfection procedures in accordance with preferred embodiments of the present invention. The Lipincott Procedures include steps and protocols for disinfection, patient care equipment, and home care. The Lipincott Procedures indicate the need for home care, including immunocompromised patients, indwelling medical devices, or communicable diseases. Improper disinfection of patient care equipment in a home environment may expose a patient to a new infection or spread an infection from one patient to another. The Lipincott Procedures classify equipment into critical, semicritical, and noncritical categories based on the degree of infection risk involved in their use. The Lipincott Procedures also include procedures configured to provide proper techniques to ensure infection control while promoting efficient use of the clinical equipment used on multiple patients.

Embodiments of the system and method described herein are configured, in part, to more orderly store equipment, supplies, tools, and patient files. For example, computer and side extensions may be utilized to input required documentation for Medicare or other patient documentation systems or standards. For example, Oasis is one such system. The system includes data entries for all care administered during the patient treatment session, including documenting insertion of a catheter, wound care, notes, billing items, etc. The standards also require the computer documentation to be completed at the administration site—i.e., at a patient's home, such that the system facilitates the ability for the practitioner to more efficiently and systematically administer care and document it appropriately. During the patient's home care session, the practitioner may administer one or more medications, or may review appropriate medications to be taken by the patient. The practitioner may draw blood for lab use. The portable printer may facilitate printing medication lists and a medication profile (that is required to be provided) and reviewing the medications and any required or needed details concerning the medications, administration of the medications, and/or any side effects. In addition, the practitioner may need to print out exercise programs, handouts, or the like, to the patient at the conclusion of a session. In addition, the practitioner may print out the handouts and other materials in advance and store them in vertical files, either with the portable printer or by using another printer. Practitioners may also more efficiently draw blood, administer medications or pharmaceuticals, and provide medication management utilizing the organization system and method disclosed herein.

One or more of the features of the system is configured to be lockable. For example, the foldable portions of the system are locking such that it maintains a stable and supported platform from which the practitioner can work. The wheels may also be lockable. The system frame may be telescoping or adjustable to facilitate various heights of the practitioner and tools, supplies, and equipment. The computer and files may also be lockable to prevent third parties from accessing or obtaining patient information.

As discussed herein the containers depicted in the system herein are configured to maintain a clean/dirty aspect such that single-use items or “dirty” objects may be taken out and sealed away separately from “clean” objects, thereby maintaining a sanitary, sterile, and safe environment for administration of nursing or medical procedures, or the like, to an in-home patient. The system and method described herein is suitable for clinicians, doctors of physical therapy, physicians, nurses, psychologists/psychiatrists, phlebotomists, or the like. The system and method is suitable for a variety of industries that relate to medical or systematized home care, including the insurance industry, that may require in-home testing prior to issuing an insurance policy. In addition, speech, respiratory, rehabilitation, and other services are envisioned herein.

The equipment, tools, and supplies are not limited to those described previously, and can include a stethoscope, wipes, gloves, hand sanitizer, cleaner and/or wipes, masks, sterile gowns, blood pressure cuffs, pulse oximeter, physical therapy equipment, and other medical, nursing, or related equipment.

The system and method avoid the practitioner from having to use the patient's tables, desks, or other platforms (that also may be themselves infected with bacteria, viruses, or other contaminants) by providing a standalone and separate organization method that does not rely on anything within a patient's home to provide the necessary services in accordance with preferred standards or procedures (i.e., Lipincott procedures, or the like).

In an embodiment, the laptop computer and printer are configured to be connected to the internet and may utilize web-based, cloud-based, or standalone services or applications in connection with the foregoing.

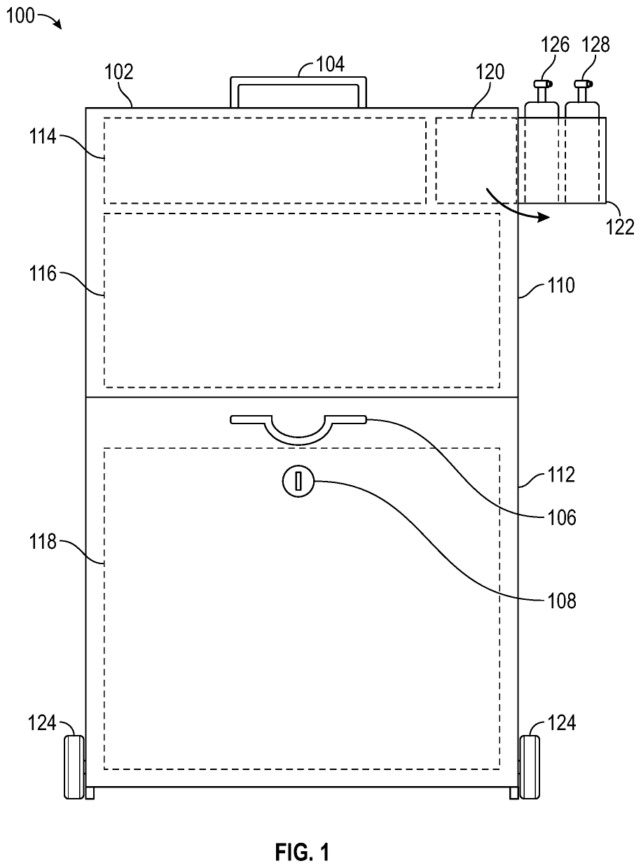

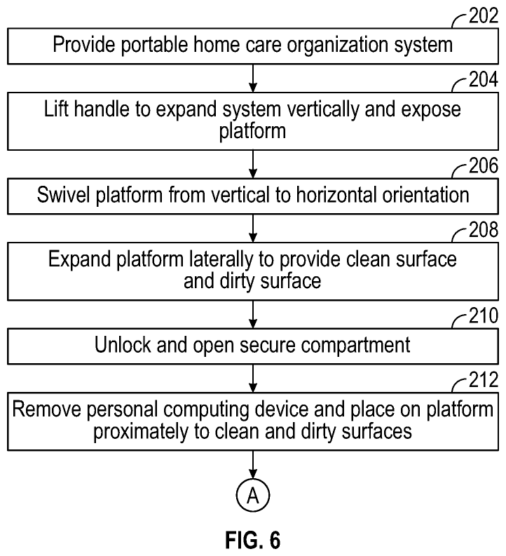

Referring now to the drawings, which are for purposes of illustrating the present invention and not for purposes of limiting the same, the drawings show devices and components (and related methods) therein in accordance with preferred embodiments of a portable home care organization system and related methods. As shown in FIGS. 1-5, the portable home care organization system generally includes an apparatus including multiple compartments and surfaces specifically configured for a medical practitioner to administer care to a patient in the patient's home or other facility in which the patient is located. As shown in FIGS. 6-8, portable home care organization methods are shown to facilitate medical care to a patient in the patient's home by a medical practitioner while complying with standardized cleanliness and sterility procedures. The portable home care organization system and methods disclosed herein assist in patient safety and help prevent or eliminate infections and pathogens from spreading to the patient and transmission of such infections or pathogens to other patients.

Referring now to FIG. 1, a front view diagram of a portable home care organization system in accordance with a preferred embodiment of the present invention is depicted. The portable home care organization system 100 depicted in FIG. 1 includes a housing 102, a first handle 104, a second handle 106, a lock 108, an upper assembly 110, a lower assembly 112, a first compartment 114, a second compartment 116, a third compartment 118, a fourth compartment 120, a soap and sanitizer holder 122, and wheels 124. The housing 102 includes internal structure (not shown) to provide support and stability for the aforementioned components. The housing 102 comprises the upper assembly 110 and the lower assembly 112. The first handle 104 is affixed to the upper assembly 110 and configured to allow a user to carry the system 100 into a patient's home. Other embodiments may utilize different types of handles or carrying mechanisms than the first handle 104 shown in FIG. 1. The first handle 104 is also configured to wheel the system 100 from a vehicle into the patient's home. The second handle 106 is affixed to the lower assembly 112 and is used to extend the lower assembly 112 to obtain access to the third compartment 118. In various embodiments, the lower assembly 112 is extendable outward by pulling on the second handle 106 by use of internal slide rails on the upper portion or lower portion of the lower assembly or internal hinges located on the lower portion of the lower assembly 112 proximate to the wheels. Other embodiments are within the scope of the present invention so long as access to the third compartment 118 is permitted. In addition, other embodiments do not include a second handle 106 as shown in FIG. 1, but may include a different type of handle or opening mechanism to gain access to the third compartment 118. The lock 108 is affixed to the outside of the lower assembly 112 and locks the lower assembly to restrict access to the third compartment 118. While the lock 108 is shown as including a keyhole, other keyless locks are within the scope of the present invention, such as, for example, a combination lock or a biometric lock.

The upper assembly 110 preferably includes structure and support (not shown) to secure the first compartment 114, the second compartment 116, and the fourth compartment 120. The first compartment 114 (e.g., a clean material compartment 114) preferably includes partitions to store clean equipment such as a blood pressure cuff, a stethoscope, a bandage scissors, a reusable tourniquet, a thermometer, a pulse oximeter, and a writing instrument. The partitions may be selected by the medical practitioner to segregate the bandage scissors and writing instruments, for example, from other clean equipment such as the blood pressure cuff, stethoscope, etc. Other clean equipment is within the scope of the present invention. As described herein, “clean” equipment is sterile or previously cleaned equipment that may be used by a medical practitioner providing home care to a patient. For example, a medical practitioner typically will measure the patient's blood pressure, blood oxygen content, heart rate, lung function, temperature, or other vitals during a home care visit. A medical practitioner may also draw blood for lab use and may require a reusable tourniquet and bandage scissors. One of ordinary skill in the art would understand that a variety of home care techniques and administration are within the scope of the present invention. The first compartment 114 is preferably sealed by a zipper, ziplock-type closure, or other suitable sealing technique to limit exposure to outside factors including airborne pathogens, and the like.

The second compartment 116 (e.g., a clean equipment compartment 116) preferably includes partitions to store clean material such as wipes, gloves, barriers, single-use disposable items, syringes, needles, masks, and personal protective equipment (PPE). The partitions may be selected by the medical practitioner to segregate the needles and syringes, for example, from other clean material such as the gloves, barriers, masks, etc. As with the clean equipment, the clean material is sterile or previously cleaned material that may be used by a medical practitioner providing home care to a patient. Similarly to the clean equipment, for example, a medical practitioner may require gloves, masks, and other PPE to ensure patient safety while providing home care. The medical practitioner may use needles and syringes to draw blood for lab use. The second compartment 116 is preferably sealed by a zipper, ziplock-type closure, or other suitable sealing technique to limit exposure to outside factors including airborne pathogens, and the like. The first and second compartments 114, 116 preferably include removable containers (not shown) that are composed of plastic or other material suitable for washing. According to standardized procedures, it is recommended to thoroughly clean and sanitize all materials of a home care bag brought into a patient's home. The compartments 114, 116 preferably are configured to be cleaned and sanitized outside of the system 100. The compartments 114, 116 also preferably include a locking mechanism such that only one of the first compartment 114 or the second compartment 116 is able to be opened at one time. For example, the compartments 114, 116 each include a mechanical locking mechanism or electronic locking mechanism preventing access to both of the compartments 114, 116 at the same time. This ensures prevention of cross-contamination between compartments 114, 116.

The lower assembly 112 preferably includes structure and support (not shown) to secure the third compartment 118 (e.g., a patient file and administration compartment 118). As discussed herein, the third compartment 118 is capable of being locked with the lock 108 such that patient files and a personal computing device such as a laptop or tablet are unable to be accessed except by the practitioner. The patient files may also include HIPAA-required paperwork, signature pages, or other paper files or handouts to provide to the patient during a home care services visit. The personal computing device is preferably provided for documenting the procedures and protocols of a home care visit, among other HIPAA-required items necessary to be documented during a home care services visit. A printer may also be stored in the third compartment 118 to print out forms, handouts, or the like during a visit. A separate partition in the third compartment 118 may be utilized to store patient blood vials, medical bottles, containers, or the like, before transporting these items to a laboratory for testing. Prior to being used on a patient, these items may be stored in the first compartment 114 and taken out for use during the home care services. In a preferred embodiment, the practitioner obtains a signature from the patient using the personal computing device, after which the personal computing device is cleaned and sanitized and, once work is complete, replaced in the third compartment 118.

Certain items, such as medical bottles, must be viewed and verified by a patient and entered into an electronic system using the personal computing device. In addition, practitioners may be required to obtain patient signatures and store them for later retrieval. These signature pages, after being touched by a patient, may be stored separately from “clean” patient files in a separately sealed partition of the third compartment 118.

The fourth compartment 120 preferably is located at a top portion of the upper assembly and is configured to be rotatably extended as a holder 122 for soap 126 and hand sanitizer 128. In an embodiment, the holder 122 is located near a clean surface (e.g., a first surface) and away from a dirty surface (e.g., a second surface). In practice, the practitioner is required to clean and sanitize equipment before placing the equipment back in the second container 116. Thus, the holder 122 provides efficient and quick access to the soap 126 and hand sanitizer 128 that may be used often.

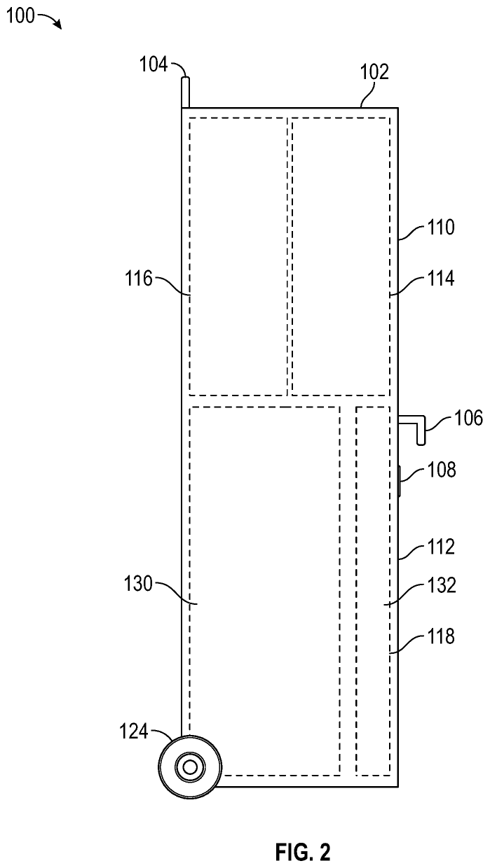

Referring now to FIG. 2, a side view diagram of a home care organization system in accordance with a preferred embodiment of the present invention is depicted. The system 100 depicted in FIG. 2 includes substantially the same elements as that depicted in FIG. 1, albeit in a different configuration. The system 100 of FIG. 2 includes a housing 102, a first handle 104, a second handle 106, a lock 108, an upper assembly 110, a lower assembly 112, a first compartment 114, a second compartment 116, a third compartment 118, and wheels 124. While not shown, the system 100 of FIG. 2 may also include a fourth compartment 120, a holder 122, and soap 126 and hand sanitizer 128.

The upper assembly shows a different vertically oriented first and second compartments 114, 116. This may be preferable to horizontally oriented first and second compartments 114, 116. The system 100 of FIG. 2 also includes a file organizer 130 and a personal computing device slot 132. As described above, the file organizer 130 may be utilized to store patient files, or the like. The slot 132 may be utilized to store a laptop or table, etc.

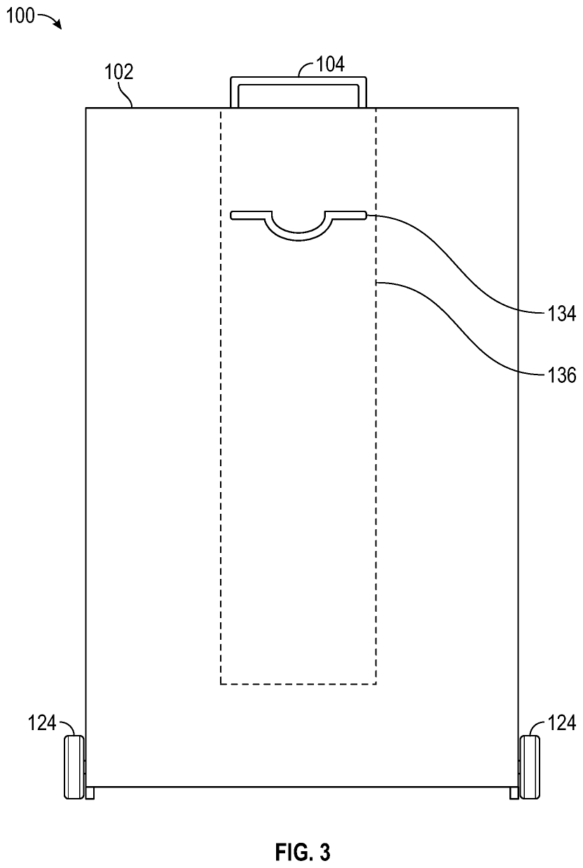

Referring now to FIG. 3, a rear view diagram of a home care organization system in accordance with a preferred embodiment of the present invention is depicted. The system 100 depicted in FIG. 3 includes substantially the same elements as that depicted in FIGS. 1 and 2, though only certain of these elements are shown. The system 100 of FIG. 3, however, includes a third handle 134 and a fifth compartment 136. The fourth compartment 136 in a preferred embodiment is utilized to store “dirty” materials or waste used by the practitioner during the home care services visit. The fifth compartment 136 permits the practitioner not having to rely on the patient's trash receptacles and is separate and sealable from the clean compartments 114, 116, 118 in the front of the system 100, in a preferred embodiment, thus reducing or eliminating the transmission of infectious disease or pathogens. In another embodiment, the fifth compartment 136 is a sharps container configured to store syringes or needles, or is a combination of a sharps container and a waste container. The patient may also already have a sharps container available in the patient's home for use by the practitioner. The sharps container may be configured to be removable so that it may be stored in a safe location of the practitioner's vehicle during transport.

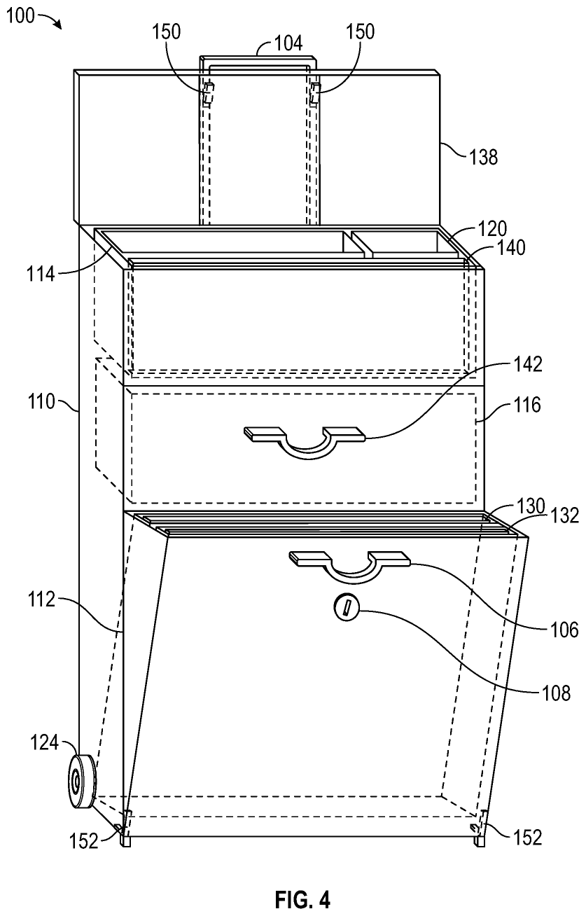

Referring now to FIG. 4, a perspective view of a partially opened home care organization system in accordance with a preferred embodiment of the present invention is depicted. The system 100 depicted in FIG. 4 includes some of the elements as that depicted in FIGS. 1-3; however, additional features and components are shown, as disclosed herein. The system 100 of FIG. 4 includes an extendible platform 138 that is configured to be extended vertically from within the upper assembly 110. For example, the platform 138 is extendible by pulling upward on the first handle 104. As shown in FIG. 4, the first handle 104 includes an extendible portion and platform hinges 150 to move or swivel the platform 138 from a vertical to a horizontal orientation. The platform 138 is further configured to be laterally extended to provide a wider working platform for the practitioner.

The system 100 of FIG. 4 also includes a stand 140 configured to provide a separate working platform. This may be suitable for placing a laptop for entry of data or information relating to a home care visit. In other embodiments, the stand 140 is a first surface or a second surface. The system 100 also includes a fourth handle 142 configured to slide out the second compartment 116 to gain access to clean equipment or materials.

The system 100 also shows a different configuration of the lower assembly 112. The lower assembly 112 in an embodiment is swiveled outward to gain access to the file organizer 130 and the slot 132. Lower assembly hinges 152 are utilized to effect the swiveling or rotating action of the lower assembly 112.

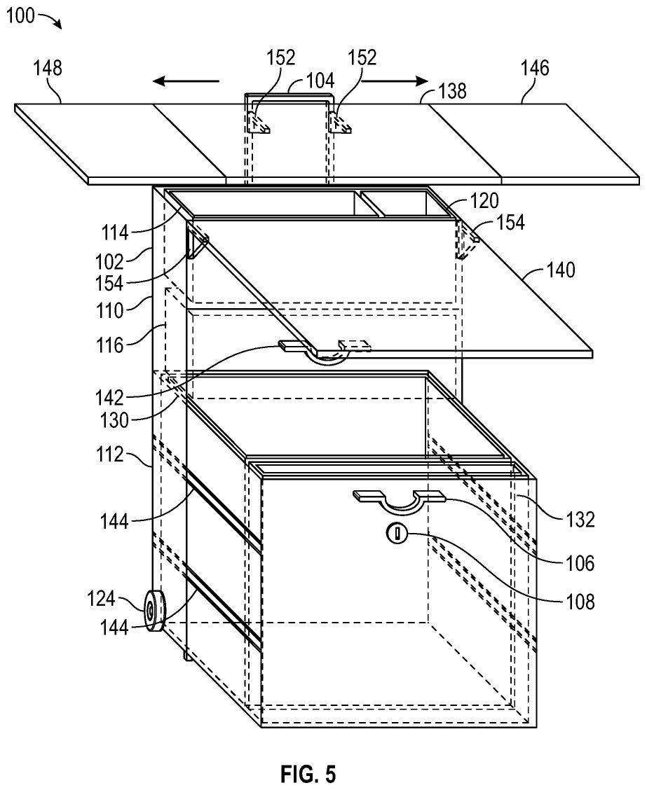

Referring now to FIG. 5, a perspective view of a partially opened home care organization system in accordance with a preferred embodiment of the present invention is depicted. The system 100 depicted in FIG. 5 includes some of the elements as that depicted in FIGS. 1-4; however, additional features and components are shown, as disclosed herein. The system 100 of FIG. 5 shows the platform 138 in a vertically extended, horizontally oriented, and laterally extended configuration. Arrows shown in FIG. 5 depict the lateral extension of the platform 138. In that configuration, the platform 138 includes a first surface 146 configured to receive clean equipment and material, and the second surface 148 configured to receive dirty equipment and material. As described herein, the first and second surfaces 146, 148 are physically separate from each other to prevent or eliminate infectious disease or pathogen transfer, and eliminate the need to use patient's belongings or surfaces during the home care services visit. The hinges 152 are shown in an extended configuration and are preferably lockable. The stand 140 is shown in a horizontally extended configuration and are extendable utilizing the stand hinges 154. The hinges 154 are shown in an extended configuration and are preferably lockable.

The lower assembly 112 as shown in FIG. 5 is differently configured from that shown in FIG. 4. In FIG. 5, the lower assembly includes slide rails 144 that are affixed to the lower assembly 112 and configured to slide out the third container 118, the file organizer 130, and the slot 132.

Referring now to FIG. 6, a flowchart of a home care organization method in accordance with a preferred embodiment of the present invention is depicted. While the method 200 disclosed herein shows steps in a particular order, certain steps may be switchable without departing from the scope of the present invention. At step 202, a portable home care organization system 100 is provided. The system 100 preferably includes the elements as disclosed in FIGS. 1-5. At step 204, the handle is lifted (e.g., vertically extended) to expand the system 100 vertically and to expose the platform 138. At step 206, the platform 138 is swiveled from a vertical to a horizontal orientation. Preferably, the platform 138 is locked into place in the horizontal orientation.

At step 208, the platform 138 is extended laterally to provide a clean surface (e.g., a first surface 146) and a dirty surface (e.g., a second surface 148). As described herein, the clean surface 146 receives clean material and equipment from the first and second compartments 114, 116, and the dirty surface 148 receives dirty material and equipment after use with the patient. The dirty equipment is cleaned and sanitized and left to dry on the clean surface 146. Once dry, the cleaned and sanitized equipment is replaced in the second compartment 116.

At step 210, the secure compartment (e.g., the third compartment 118) is unlocked and opened to provide access to the patient's files and personal computing device in the file organizer 130 and the slot 132, respectively. At step 212, the personal computing device is removed from the secure compartment 118 (e.g., from the slot 132) and placed on the platform 138 proximately to the clean and dirty surfaces 146, 148. Alternatively, the device is placed on the stand 140.

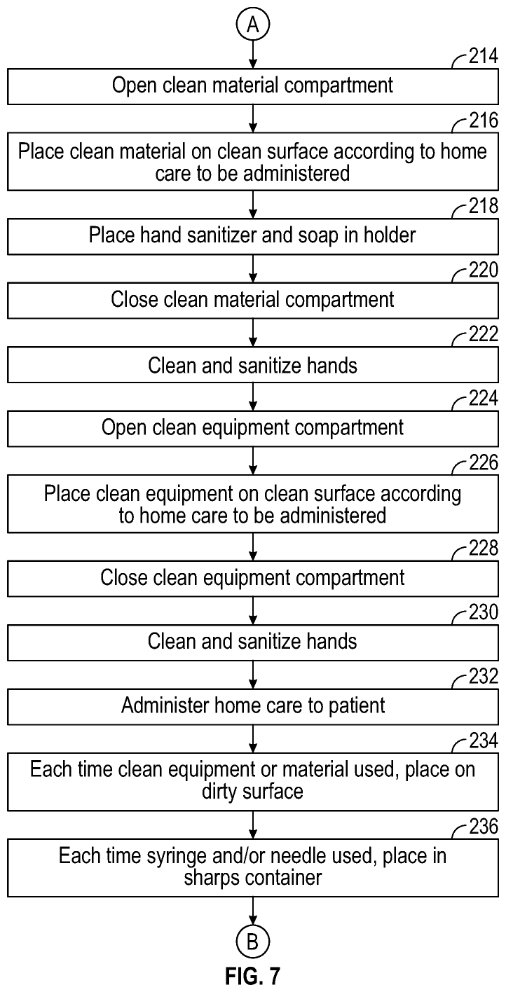

FIG. 7 is a continuation of the method 200. At step 214, the clean materials compartment (e.g., the first compartment 114) is opened. At step 216, clean material (e.g., as described herein) is placed on the clean surface 146 according to the home care to be administered. For example, the practitioner may be drawing blood for use in a lab, and may require a syringe, a needle, a bandage, etc., to administer the treatment. Those pieces of equipment are placed on the clean surface 146.

At step 218, the soap 126 and hand sanitizer 128 are placed in the holder 122. At step 220, the clean material compartment 114 is closed. At step 222, the practitioner's hands are cleaned and sanitized. These steps are typically required to prevent the spread of infectious disease and pathogens to the patient or elements of the system 100.

At step 224, the clean equipment compartment (e.g., the second compartment 116) is opened. At step 226, clean equipment (e.g., as described herein) is placed on the clean surface 146 according to the home care to be administered. For example, the practitioner may be measuring vital signs such as heartbeat, lung function, blood oxygen levels, or the like, and may require a stethoscope, pulse oximeter, and the like, to administer the treatment. Those pieces of equipment are placed on the clean surface 146. At step 228, the clean material compartment 114 is closed. At step 230, the practitioner's hands are cleaned and sanitized. These steps are typically required to prevent the spread of infectious disease and pathogens to the patient or elements of the system 100.

At step 232, the home care services are administered to the patient. As described herein, a variety of home care services or treatment is within the scope of the present invention. Relevantly, the administration of services requires that equipment and material touch the patient, which may then carry infectious disease, pathogens, or the like, and become “dirty” equipment and material. At step 234, therefore, each time that clean equipment is used on the patient, that equipment is placed on the dirty surface 148. At step 236, each time that a syringe and/or needle is used, that equipment is placed in a sharps container in accordance with applicable procedures, guidelines, and requirements.

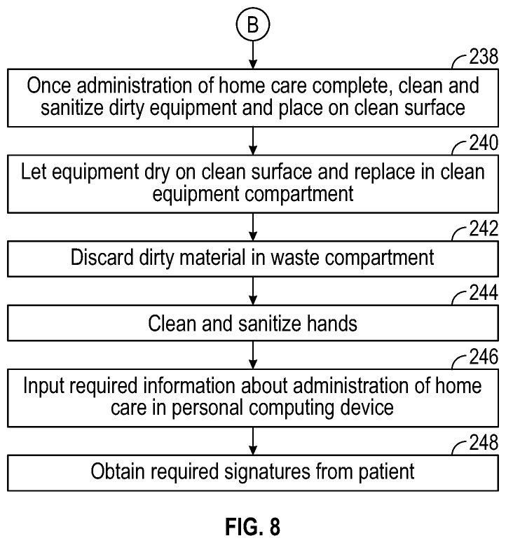

FIG. 8 is a continuation of the method 200. At step 238, the dirty equipment is cleaned and sanitized and placed on the clean surface 146 once administration of the home care services is completed. At step 240, the equipment is left to dry on the clean surface 146, and once dry, is replaced in the clean equipment compartment 116. The drying process may take 2-5 minutes. Once dry, the equipment is considered clean and may be replaced in the clean equipment compartment 116.

At step 242, the dirty material is discarded in the waste compartment (e.g., the fifth compartment 136). As disclosed herein, the waste compartment 136 may include a separate sharps container for disposal of sharps such as syringes or needles. At step 244, the practitioner's hands are cleaned and sanitized. These steps are typically required to prevent the spread of infectious disease and pathogens to the patient or elements of the system 100.

At step 246, required information about the administration of the home care services are inputted using the personal computing device. Practitioners practicing home care services must comply with HIPAA and other guidelines and requirements. While the current method 200 envisions electronic entry, other suitable forms of data entry are within the scope of the present invention. At step 248, the required signatures from the patient are obtained. Oftentimes at the close of a home care visit, practitioners are required to obtain signatures of the patient for their confirmation that certain activities were conducted during the visit. Other times, consent to procedures are necessary before or during a visit; in those circumstances, the practitioner must obtain those signatures at those other times and thus, may be obtained at different times than that identified in the method 200. Further steps for cleaning and sanitizing writing instruments and/or signature pages are disclosed herein. In a preferred embodiment, the patient signatures are obtained through the personal computing device stored in the third compartment 118, after which it is cleaned and sanitized by the practitioner and replaced in the third compartment 118.

In a preferred embodiment of the present invention, functionality is implemented as software executing on a server that is in connection, via a network, with other portions of the system, including databases and external services. The server comprises a computer device capable of receiving input commands, processing data, and outputting the results for the user. Preferably, the server consists of RAM (memory), hard disk, network, central processing unit (CPU). It will be understood and appreciated by those of skill in the art that the server could be replaced with, or augmented by, any number of other computer device types or processing units, including but not limited to a desktop computer, laptop computer, mobile or tablet device, or the like. Similarly, the hard disk could be replaced with any number of computer storage devices, including flash drives, removable media storage devices (CDs, DVDs, etc.), or the like.

The network can consist of any network type, including but not limited to a local area network (LAN), wide area network (WAN), and/or the internet. The server can consist of any computing device or combination thereof, including but not limited to the computing devices described herein, such as a desktop computer, laptop computer, mobile or tablet device, as well as storage devices that may be connected to the network, such as hard drives, flash drives, removable media storage devices, or the like.

The storage devices (e.g., hard disk, another server, a NAS, or other devices known to persons of ordinary skill in the art), are intended to be nonvolatile, computer readable storage media to provide storage of computer-executable instructions, data structures, program modules, and other data for the mobile app, which are executed by CPU/processor (or the corresponding processor of such other components). The various components of the present disclosure, are stored or recorded on a hard disk or other like storage devices described above, which may be accessed and utilized by a web browser, mobile app, the server (over the network), or any of the peripheral devices described herein. One or more of the modules or steps of the present disclosure also may be stored or recorded on the server, and transmitted over the network, to be accessed and utilized by a web browser, a mobile app, or any other computing device that may be connected to one or more of the web browser, mobile app, the network, and/or the server.

References to a “database” or to “database table” are intended to encompass any system for storing data and any data structures therein, including relational database management systems and any tables therein, non-relational database management systems, document-oriented databases, NoSQL databases, or any other system for storing data.

Software and web or internet implementations of the present disclosure could be accomplished with standard programming techniques with logic to accomplish the various steps in accordance with the present disclosure described herein. It should also be noted that the terms “component,” “module,” or “step,” as may be used herein, are intended to encompass implementations using one or more lines of software code, macro instructions, hardware implementations, and/or equipment for receiving manual inputs, as will be well understood and appreciated by those of ordinary skill in the art. Such software code, modules, or elements may be implemented with any programming or scripting language such as C, C++, C#, Java, Cobol, assembler, PERL, Python, PHP, or the like, or macros using Excel or other similar or related applications with various algorithms being implemented with any combination of data structures, objects, processes, routines or other programming elements.

The above-detailed description of embodiments of the disclosure is not intended to be exhaustive or to limit the teachings to the precise form disclosed above. While specific embodiments of and examples for the disclosure are described above for illustrative purposes, various equivalent modifications are possible within the scope of the disclosure, as those skilled in the relevant art will recognize. For example, while processes or blocks are presented in a given order, alternative embodiments may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are at times shown as being performed in series, these processes or blocks may instead be performed in parallel, or may be performed, at different times. Further any specific numbers noted herein are only examples: alternative implementations may employ differing values or ranges. It will be appreciated that any dimensions given herein are only exemplary and that none of the dimensions or descriptions are limiting on the present invention.

The teachings of the disclosure provided herein can be applied to other systems, not necessarily the system described above. The elements and acts of the various embodiments described above can be combined to provide further embodiments.

Any patents and applications and other references noted above, including any that may be listed in accompanying filing papers, are incorporated herein by reference in their entirety. Aspects of the disclosure can be modified, if necessary, to employ the systems, functions, and concepts of the various references described above to provide yet further embodiments of the disclosure.

These and other changes can be made to the disclosure in light of the above Detailed Description of the Preferred Embodiments. While the above description describes certain embodiments of the disclosure, and describes the best mode contemplated, no matter how detailed the above appears in text, the teachings can be practiced in many ways. Details of the system may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features or aspects of the disclosure with which that terminology is associated. In general, the terms used in the following claims should not be construed to limit the disclosures to the specific embodiments disclosed in the specification unless the above Detailed Description of the Preferred Embodiments section explicitly defines such terms. Accordingly, the actual scope of the disclosure encompasses not only the disclosed embodiments, but also all equivalent ways of practicing or implementing the disclosure under the claims.

While certain aspects of the disclosure are presented below in certain claim forms, the inventors contemplate the various aspects of the disclosure in any number of claim forms. For example, while only one aspect of the disclosure is recited as a means-plus-function claim under 35 U.S.C. § 112, ¶16, other aspects may likewise be embodied as a means-plus-function claim, or in other forms, such as being embodied in a computer-readable medium. (Any claims intended to be treated under 35 U.S.C. § 112, ¶6 will begin with the words “means for”). Accordingly, the applicant reserves the right to add additional claims after filing the application to pursue such additional claim forms for other aspects of the disclosure.

Accordingly, although exemplary embodiments of the invention have been shown and described, it is to be understood that all the terms used herein are descriptive rather than limiting, and that many changes, modifications, and substitutions may be made by one having ordinary skill in the art without departing from the spirit and scope of the invention.

1. A home care organization system comprising:
a housing,
an upper assembly comprising a first compartment, wherein the first compartment is configured to store clean equipment and material,
a platform configured to be movable from a vertical to a horizontal orientation relative to the upper assembly, wherein the platform is laterally extendable to provide a first surface configured to receive clean equipment and material and a second surface configured to receive dirty equipment and material, and
a lower assembly.

2. The home care organization system of claim 1 wherein the first compartment is sealable.

3. The home care organization system of claim 1 wherein the first compartment is segregated into a first partition and a second partition, the first partition configured to store the clean material and the second partition configured to store the clean equipment.

4. The home care organization system of claim 1 wherein the upper assembly further comprises a second compartment configured to store the clean equipment, and wherein the first compartment is configured to store the clean material.

5. The home care organization system of claim 4 wherein the upper assembly further comprises a locking mechanism such that only one of the first compartment or the second compartment is able to be opened at one time.

6. The home care organization system of claim 1 wherein the housing comprises a handle configured to expand the upper assembly vertically to position the platform at a specified height from the bottom of the housing.

7. The home care organization system of claim 1 wherein the clean equipment comprises one or more of a blood pressure cuff, a stethoscope, a bandage scissors, a reusable tourniquet, a thermometer, a pulse oximeter, and a writing instrument.

8. The home care organization system of claim 1 wherein the clean material comprises one or more of wipes, gloves, barriers, single-use disposable items, syringes, needles, masks, and personal protective equipment.

9. The home care organization system of claim 1 wherein the lower assembly comprises a third compartment configured to store one or more of patient files, a personal computing device, and a printer, and a lock configured to secure the third compartment.

10. The home care organization system of claim 1 wherein the housing includes a front portion and a rear portion, wherein the rear portion comprises a third compartment configured to store dirty material.

11. The home care organization system of claim 1 wherein the upper assembly comprises a fourth compartment configured to swivel out laterally from the upper assembly and to receive one or more of hand sanitizer and soap.

12. The home care organization system of claim 1 wherein the upper assembly comprises a fifth compartment configured to store dirty material.

13. The home care organization system of claim 1 wherein the upper assembly comprises a sixth compartment configured to store a sharps container.

14. The home care organization system of claim 1 further comprising lockable wheels affixed to the lower assembly.

15. The home care organization system of claim 1 wherein the platform is configured to receive a personal computing device.

16. A home care organization method, the method comprising the steps of:
providing a home care organization system including a housing, an upper assembly comprising first and second compartments, wherein the first compartment is configured to store clean material and the second compartment is configured to store clean equipment, a platform configured to be movable from a vertical to a horizontal orientation relative to the upper assembly, wherein the platform is laterally extendable to provide a first surface configured to receive clean equipment and material and a second surface configured to receive dirty equipment and material, and a lower assembly, wherein the lower assembly comprises a third compartment configured to store patient files and a personal computing device,
expanding the system vertically to expose the platform,
moving the platform from to a horizontal orientation relative to the upper assembly,
expanding the platform laterally to provide the first surface and the second surface,
removing and placing the personal computing device on the platform proximate to the first and second surfaces,
providing home care services to a patient using the clean material and equipment, the platform, including the first and second surfaces, and the personal computing device.

17. The method of claim 16 further comprising
opening the first compartment to access the clean materials,
placing the clean material on the first surface in accordance with the home care services to be administered to the patient,
closing the first compartment, and
cleaning and sanitizing hands used to provide the home care services.

18. The method of claim 16 further comprising
opening the second compartment to access the clean equipment,
placing the clean equipment on the first surface in accordance with the home care services to be administered to the patient,
closing the second compartment, and
cleaning and sanitizing hands used to provide the home care services.

19. The method of claim 16 wherein when the home care services are provided to the patient, placing used material and equipment on the second surface to isolate the used material and equipment from the clean material and equipment.

20. The method of claim 19 further comprising
cleaning and sanitizing the used equipment,
placing cleaned and sanitized equipment on first surface,
replacing the cleaned and sanitized equipment in the second compartment,
discarding the used material in a fourth compartment configured to handle waste.

21. The method of claim 16 further comprising
inputting information documenting the home care services using the personal computing device.

22. The method of claim 16 further comprising
obtaining a signature of the patient consenting to the home care services using the personal computing device,
cleaning and sanitizing the personal computing device, and
replacing the personal computing device in the third compartment.

23. The method of claim 16 wherein the clean material includes at least one of a syringe and needle, and wherein when the at least one of the syringe and needle are used, placing the used syringe and needle into a sharps container.

24. The method of claim 16 further comprising
placing hand sanitizer and soap in a holder rotatably connected to the upper assembly.
